(19) United States
(12) Patent Application Publication
(10) Pub. No.: US 2005/0126578 A1
(43) Pub. Date: Jun. 16, 2005

(54) External pressure garment in combination with a complementary positive pressure ventilator for pulmocardiac assistance

(76) Inventors: Richard L. Garrison, Houston, TX (US); Tariq A. Khan, Houston, TX (US)

Correspondence Address: TARIQ KHAN, 2322 SAXON DRIVE, HOUSTON, TX, 77018, US

(21) Appl. No.: 10/735,792

(22) Filed: Dec. 12, 2003

(51) Int. Cl. A61F 5/37

(52) U.S. Cl. 128/874

(57) ABSTRACT

A pulmocardiac assistance apparatus is disclosed for use in the medical technology field to provide expiratory and inspiratory functionality to patients with respiratory disorders. The pulmocardiac assistance device of the present invention functions to collect body response feedback and manages this feedback to achieve an optimum response. The device includes an integrated set of pressure cuffs for pressuring different parts of the body in accordance with a predetermined sequence to induce the patient to breathe out or expire air from the lungs or to even produce an assisted cough, as well as to promote circulation of blood from the extremities of the body to the head. The pulmocardiac assistance device further includes a programmable logic controller which accepts inputs from sensors (e.g. patient temperature, blood gas concentrations, arterial pH level) and makes calculations to control both ventilator and pressure cuffs.

### BACKGROUND OF THE INVENTION

1. Field of the Invention

The present invention relates generally to the medical respiratory field, and more particularly, to a system for assisting expiratory/inspiratory functions and blood circulation in a human patient.

2. Description of the Prior Art

Currently, known devices that are capable of facilitating both inspiratory and expiratory operations in the human body to combat respiratory disorders include: the iron lung, the cuirass, and the pneumo-wrap. However, these devices are extremely restrictive and cumbersome and provide only limited access to the body of the patient. This hinders the ability of health care professionals to treat the patient as the only access to the patient's body for treatment is via various portholes in the device, or via temporary removal of the device, which results in loss of ventilatory effectiveness during access or removal. Also, this limited access to the patient's body creates personal hygiene problems for the patient. Moreover, while the iron lung, the cuirass, and the pneumo-wrap provide somewhat satisfactory expiratory functionality to the patient, their inspiratory functionality is less satisfactory. For these reasons, the positive pressure ventilator has all but replaced the use of these devices for personal respiratory assistance.

Yet, while the positive pressure ventilator is a superior inspiratory system, it is unable to provide any expiratory assistance. Considering that expiratory disorders account for many cases of respiratory failure leading to assisted ventilation, this is a significant problem. Indeed, attempts to empty the lungs via negative endotracheal pressure lead to collapse of airways rather than efflux of air. Therefore, assistance with expiration should be externally applied to the thoracic wall. The iron lung, pneumo-wrap, and cuirass do facilitate expiration via external pressure, but are not much used for the reasons cited above.

Other common problems caused by ventilator-assisted inspiration include pneumothorax and impaired cardiac output. These two problems are associated with high intraalveolar pressures associated with positive pressure ventilation. These problems, however, can be ameliorated through externally applied assisted expiration.

Accordingly, there is a need in the medical respiratory field for an assisted respiratory device that satisfactorily combines maximally efficient (that is, positive pressure) assisted inspiration with maximally efficient (that is, externally applied pressure) assisted expiration in a practical manner that also effectively addresses the current problems with patient access and cardiac output. The present invention meets that need.

### SUMMARY OF THE INVENTION

In accordance with the present invention a method and apparatus for assisting respiratory functions in a human patient is provided. Particularly, the method and apparatus are used to facilitate expiratory functions of respiration in a human patient.

One object of the present invention is to provide an external pressure system which collects bodily response feedback and manages such feedback to achieve an optimum response. This system includes an external pressure garment which allows ready access to the body of the patient for medical and for personal hygienic concerns. The external garment includes a set of pressure cuffs comprising: (1) forearm cuffs, extending nearly from wrist to elbow, (2) lower leg cuffs, extending almost from ankle to knee, (3) upper arm cuffs, extending nearly from elbow to axilla (4) thigh cuffs, extending nearly from knee to groin, (5) an abdominal cuff, extending roughly from pubis to subcostal margin, and (6) a breast-conforming thoracic cuff, extending from the subcostal margin to the infraclavicular area, containing embedded cutaneous electrical contacts for electrocardiography and defibrillation, and containing embedded piezo or other vibrating devices for pulmonary toilet. Migration of these cuffs is prevented by shoulder straps on the thoracic cuff, ankle stirrups in the lower leg cuffs, and straps connecting all cuffs to each other. Alternatively, the set of pressure cuffs may include: a first torso cuff, extending from an upper terminus just below the armpits to a lower terminus beneath the diaphragm; and/or a second torso cuff, extending from an upper terminus near the diaphragm to a lower terminus just above the groin. Each pressure cuff includes a set of channels (e.g., bladders or chambers) for receiving a temperature-conditioned pressurizing medium—such as gas, electroexpansile gel or fluid. When it receives the gas, gel or fluid (via a drive system), the set of channels expands to apply pressure to the body part enclosed by that cuff. The cuffs, in another embodiment, could be made of an elastic material, wrapped around the body part and then pulled to exert pressure or released to decrease pressure. This embodiment would not be as effective as one using expandable channels, chambers, or bladders.

The system further includes: (1) a heated and cooled reservoir for storing the gas, fluid or electroexpansile gel at any chosen temperature to fill the set of bladders of each cuff, (2) a drive system (e.g., a pump) to supply the gas, fluid, or electroexpansile gel to the cuffs, (3) a set of lines for bringing the gas, fluid or electroexpansile gel from the reservoir (via the pump) to the bladders of each cuff, (4) a processing device such as a valve system for controlling the flow of the gas, fluid or electroexpansile gel to each respective cuff, (5) a programmable logic controller (PLC) that accepts inputs from the various sensors and makes calculations used to control the processing device and the pump, (6) manual and automatic thermostatic controls (including but not limited to various inputs and logic controls and including provision for deliberate hypothermia and/or hyperthermia and appropriate responses to fever) for the heating and cooling of the gas, fluid or electroexpansile gel, (7) manual and automatic software and hardware linkages between the system and a coordinated positive pressure inspiratory ventilator (including but not limited to various inputs and logic controls) to allow control of aerosolized pharmaceuticals for control of airway resistance and automatic control of respiratory rate, tidal volume, and inspired oxygen concentration to achieve desired arterial pH and blood gas concentrations, (8) an automatic and manually-controlled side-port suction-catheter-equipped endotracheal tube, and (9) cough and/or breath-holding controls.

In operation, the PLC directs the sequence in which the cuffs pressure up by opening the valves to supply the cuffs with gas, fluid or electroexpansile gel. This sequence is as follows: (1) the forearm cuffs and lower leg cuffs pressure up, (2) the upper arm cuffs and thigh cuffs pressure up, (3) the abdominal cuff pressures up, and (4) the breast-conforming thoracic cuff pressures up. This particular sequence of applying pressure to different parts of the body induces the patient to breathe out or expire air from the lungs, as well as promotes circulation of blood from the extremities of the body (i.e., arms and legs) to the head. Moreover, use of these pressure cuffs in accordance with the present invention limits physical coverage of the body of the patient such that medical practitioners have convenient access for treatment of wounds and insertion of intravenous and intraarterial devices into sites—such as cervical, subclavian, antecubital, hand, wrist, and femoral sites. Moreover, visual access to patients is enhanced by the availability of all cuffs in transparent material. Furthermore, access for dressing changes and other kinds of hands-on patient care is accomplished by means of removable sub-segments of each cuff. Still furthermore, where the garment is not a single piece but rather jointed (i.e., composed of separate cuffs), full and ready access to the body of the patient is provided at the head, neck, hands, feet, elbows, knees, pelvic girdle, shoulder girdle, and thoraco-abdominal junction.

Another object of the present invention is to provide efficient expiratory assistance which when combined with a positive pressure ventilator for inspiratory assistance results in a medical system by which total cybernetic respiratory control of the patient is achieved. The PLC accepts input from trancutaneous oxygen and carbon dioxide saturations, and/or from intraarterial blood gas and pH monitoring, to allow system control of respiratory rate, tidal volume, and inspired oxygen concentration. Because the PLC is updated cycle-to-cycle, variability in respiratory parameters (which is desirable) is a deliberate and intentional result. The PLC automatically compensates for metabolic acidosis or alkalosis. Because the PLC is aware of blood gases, pH, and respiratory parameters, it will output information on metabolic or respiratory alkalosis, acidosis, and compensation, and suggest appropriate differential diagnosis and/or intervention. Because the PLC is aware of both inspiratory and expiratory resistance to air flow, in cases of obstruction, it automatically suggests and/or conducts therapeutic trials with aerosolized bronchodilating pharmaceuticals dispensed from reservoirs in the positive-pressure ventilator to determine comparative effectiveness of various agents and/or combinations of agents to correct pathological reversible obstruction.

The PLC can also accept a “cough” command, a capability found in no other ventilatory assist system. Prior to an automated cough, the PLC triggers an alarm to notify a conscious patient that the cough is imminent. Then the PLC closes an “artificial glottis” while pressuring up the expiratory cycle. After sufficient pressure has been achieved, the “glottis” opens to allow the rapid efflux of air so desirable in pulmonary toilet. This cough command may be manually triggered by either the practitioner or by the patient, or may be automatically programmed to occur at the selected interval.

A similar usage facilitates “breath-holding”, a maneuver that—like coughing—is available in no other ventilatory assist system. This functionality is particularly useful when switching a subject from one pulmocardiac assist system to another which of course requires disconnection and reconnection. Breath-holding in the interim makes such transfer easier. Ordinarily the “glottis” closes at the beginning of inspiration and remains closed until the end of inspiration, opening then to allow egress of air during expiration. By holding the glottis closed at the end of inspiration, so that pressure is maintained for the selected time, “breath-holding” is accomplished. In addition to facilitating transfer from one pulmocardiac assist system to another, this also provides a maneuver for reducing movement artifact as well as electrical artifact for such things as electrocardiography.

As a further assistance in respiratory toilet, the PLC accepts acoustic input from the endotracheal tube, and when the appropriate sounds of upper airway obstruction are encountered, the PLC automatically triggers the cough command. However the cough is triggered, whether by manual command, automatic time interval, or acoustic input, the system follows up each cough with endotracheal suction. The endotracheal tube has a side port with a motor-driven, gently curved, soft suction catheter. The PLC directs the tube down the trachea and right mainstem bronchus, suctions while retracting, then rolls the catheter 180 degrees and repeats the process in the left mainstem bronchus, finally rolling the catherter back 180 degrees to its resting position. Suction may be manually triggered as well by both practitioner and patient, or triggered by specified time interval.

The endotracheal tube used with this system also has EMG pickups located bilaterally in the laryngeal region, and ventrally in the tongue region. In conjunction with EMG pickups for the face, including the lips and mandible, these pickups provide muscle activity inputs that may be used in two ways. First, the information may be directly provided to the artificial glottis and “mouth” or “voice box” so that vocalization may be directly accomplished. Secondly, the information may be sent to an electronic processor for production of a purely synthetic vocalization.

Yet another object of the present invention is to provide a breast-conforming thoracic pressure cuff including a set of electrocardiogram (EKG) leads which allows for three, twelve, or more-leaded EKG's or other heart monitoring devices. This allows the patient to be monitored for heart conditions without interfering with the expiratory assistance functionality of the thoracic cuff. Because EKG data is integral to the garment, EKG gating is a feature of garment pressurization. While arterial flow may be compromised to some extent by the pressuring-up cycle for expiration, in circumstances where it is important, gating can drive small depressurizations within each larger expiratory pressurization, timed to ameliorate the increase in cardiac preload induced by regular external pressurization.

Still another object of the present invention is to provide a thoracic pressure cuff including an integrated pair of anterior and posterior plates to facilitate defibrillation by process of applying an electronic shock to the heart. The plate may be accessed by a standard defibrillator pad, an automatic external defibrillator device, or an integrated automatic external defibrillator device.

A further object of the present invention is to provide a thoracic cuff having a set of integrated piezo vibrators or other vibration devices for facilitating pulmonary toilet.

Still a further object of the present invention is to provide body temperature control by circulating a medium (e.g. a hot/cold gas, liquid, or electrexpansive gel) through the pressure cuffs to heat or cool the body of the patient as required. The temperature control system may operate manually or automatically by relying on thermal probes positioned on or in the body of the patient. The PLC input includes both a patient-controlled default thermostat and a practitioner-controlled override, and outputs a graphical display of temperature versus heating and cooling requirements by which fever and spontaneous hypothermia may be easily appreciated. A conventional heating and/or cooling device may be connected to the medium reservoir.

Yet a further object of the present invention is to provide a cuff or set of cuffs for facilitating circulation that can be used without a garment or being attached to the other cuffs. Anchoring the cuff or cuffs could be achieved in a variety of ways, including but not limited to adhesives.

An even further object of the invention is to provide a new G-suit for use in aviation that incorporates at least the cuffs, and a modified PLC that would facilitate improved circulation to the brain for a pilot in a high-G environment. Another embodiment could allow automatic defibrillation if needed.

Still a further object of this invention is to provide for patient portability while using the respiratory or circulatory assist functions. The equipment can be mounted on a fully-configured bed, cart, wheelchair, walker or other motorized or pushed device (e.g. an automoble) to facilitate patient mobility and quality of life. Furthermore, the equipment could be integrated (via wall or other mounting) into an intensive care unit (ICU), other patient unit, or even a residence.

Other objects and features of the invention will be readily apparent from the accompanying drawing and detailed description of the preferred embodiment.

### BRIEF DESCRIPTION OF THE DRAWINGS

FIG. 1 is a full view of a typical human body fitted with pressure cuffs in accordance with the present invention.

FIG. 2 is a schematic of an external pressure garment in accordance with the present invention.

FIG. 3 is a frontal view of an open pressure cuff in accordance with the present invention.

FIG. 4 is a profile view of an open pressure cuff in accordance with the present invention.

FIG. 5 is a profile view of a closed pressure cuff in accordance with the present invention depicting the bladders deflated.

FIG. 6 is a profile view of a closed pressure cuff in accordance with the present invention depicting the bladders inflated.

FIG. 7 is a flow diagram in accordance with the present invention depicting the sequencing of the pressure cuffs and the resulting blood flow path in the human body from the extremities to the head.

FIG. 8 is an enlarged schematic of profile view of an artificial glottis apparatus to provide coughing functionality in accordance with the present invention depicting the ventilator facilitating inspiration of the patient.

FIG. 9 is an enlarged schematic of profile view of an artificial glottis apparatus to provide coughing functionality in accordance with the present invention depicting the pressure cuffs being actuated while the artificial glottis is in the closed position.

FIG. 10 is an enlarged schematic of profile view of an artificial glottis apparatus to provide coughing functionality in accordance with the present invention depicting a cough being achieved by moving the artificial glottis to the closed position thus permitting a rapid efflux of air.

FIG. 11 is an enlarged profile view of the respiratory tract of a patient depicting the insertion catheter of the present invention in the removed from the endotracheal tube.

FIG. 12 is an enlarged profile view of the respiratory tract of a patient depicting the insertion catheter of the present invention being inserted into the right mainstem bronchus of the patient via the endotracheal tube to facilitate suctioning.

FIG. 13 is an enlarged profile view of the respiratory tract of a patient depicting the insertion catheter of the present invention being rotated into the left mainstem bronchus of the patient via the endotracheal tube to facilitate suctioning.

### DESCRIPTION OF A PREFERRED EMBODIMENT OF THE PRESENT INVENTION

In the medical technology field, a pulmocardiac assistance system is used to provide expiratory and inspiratory functionality to patients with respiratory disorders. The pulmocardiac assistance system functions to collect body response feedback and manages this feedback to achieve an optimum response.

In the specification and appended claims: (1) the terms “attached,” “connected,” “connecting”, and “connection” are used to mean “in direct connection with” or “in connection with via another element” ; and (2) the term “set” is used to mean “one” or “more than one”.

A description of certain embodiments of the present invention is provided to facilitate an understanding of the invention. This description is intended to be illustrative and not limiting of the present invention.

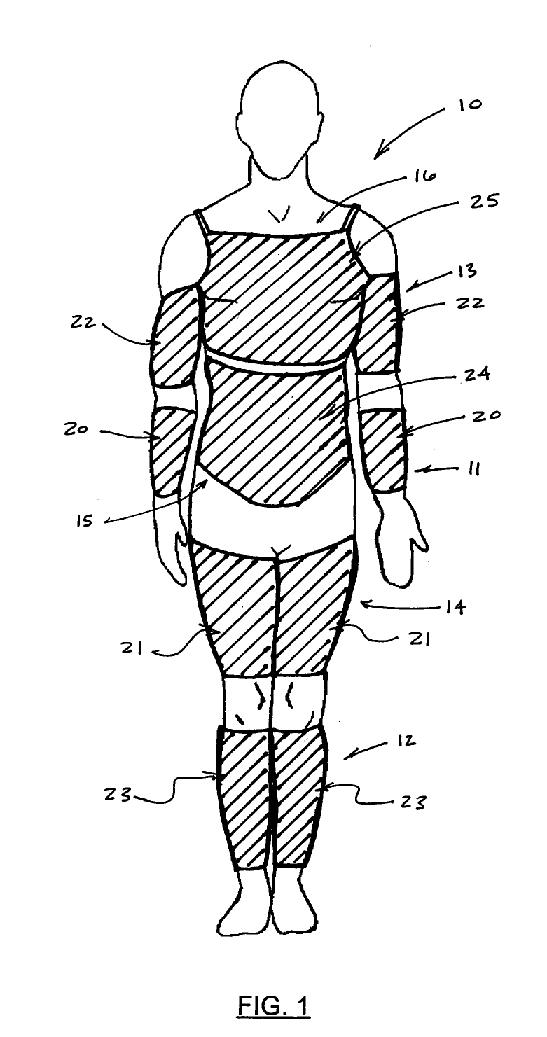

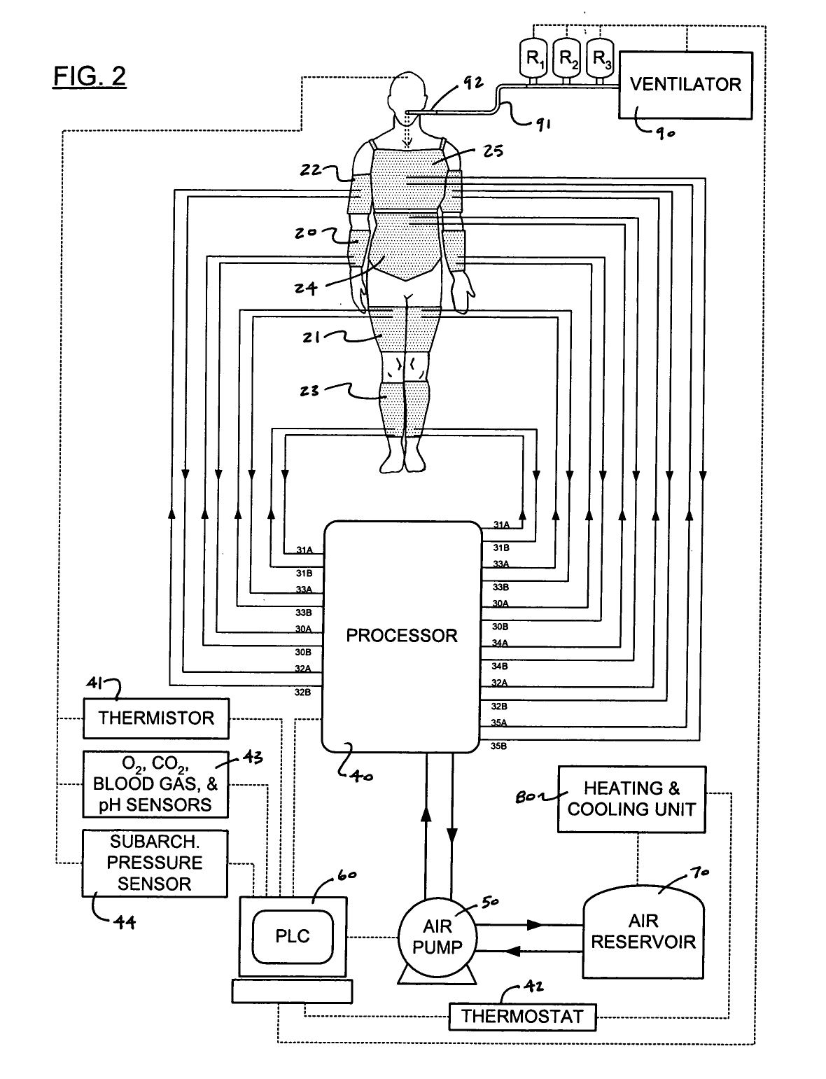

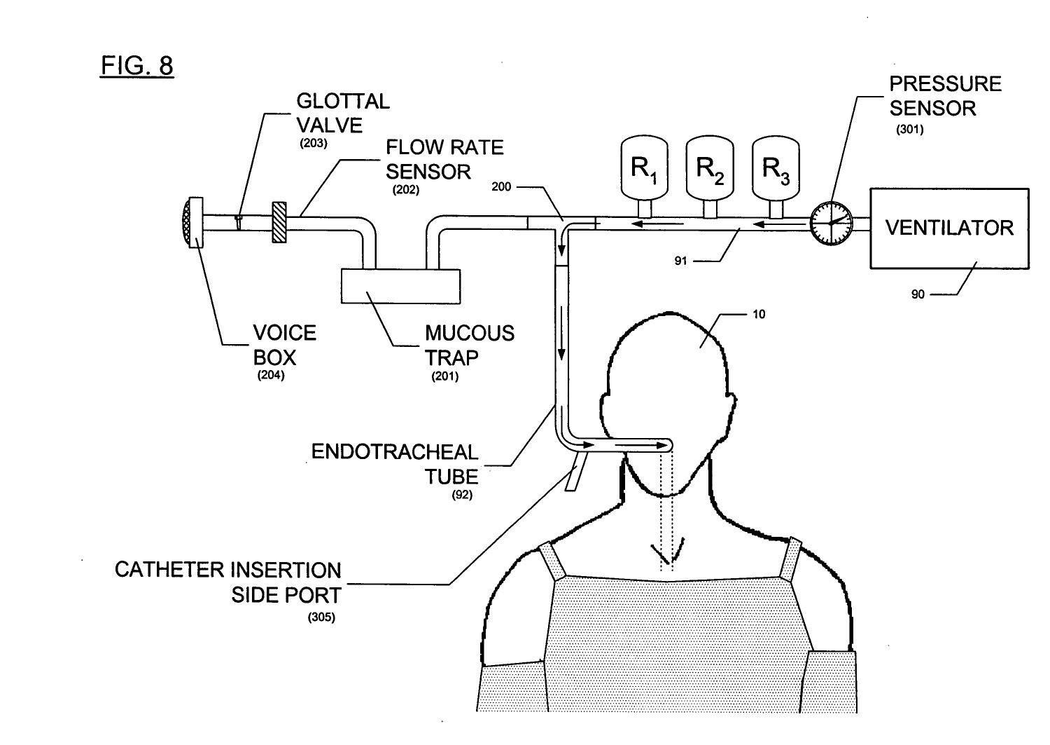

One embodiment of the present invention is a pulmocardiac assistance device for providing cybernetic inspiratory and expiratory control of a patient's respiration. With respect to FIGS. 1 and 2, the general components of a pulmocardiac assistance device in accordance with the present invention are illustrated. The pulmocardiac assistance device comprises: (1) a set of pressure cuffs 20-25 for pressurizing particular regions 11-16 of the body 10 of a patient according to a predetermined sequence; (2) a set of supply lines 30A-35A for carrying air to pressurize the set of cuffs; (3) a set of return lines 30B-35B for carrying air to depressurize the set of cuffs; (4) an air reservoir 70 for supplying and storing the air; (5) an air pump 50 for driving the air supplied to the cuffs and returned to the reservoir; (6) a central processor 40 including a set of control valves for controlling the air or fluid flow to and from the pressure cuffs in accordance with the predetermined sequence; (7) a programmable logic controller 60 for controlling any or all of the following: (a) temperature; therefore, the PLC may be connected to a thermister probe 41 and a patient-controlled thermostat 42; (b) respiratory rate, tidal volume, and inspired oxygen concentration; therefore, the PLC may be connected to transcutaneous oxygen and carbon dioxide sensors and arterial blood gas and pH sensors 43; (c) cough functionality; therefore, the PLC may be connected to a noise sensor 300 and/or pressure sensor 301 (as shown in FIGS. 8-13); (d) endotracheal suctioning; therefore, the PLC may be connected to an insertion drive 302 (e.g., twin servo motors) and a rotation drive 303 (e.g., roll control motor) for driving a suction catheter 304 through an airtight/autosealing side port 305 and rotating the suction catheter to facilitate the right mainstem bronchus and the left mainstem bronchus and a suction valve 310 connected to a vacuum reservoir 311 via a mucous trap 312 (as shown in FIGS. 8-13); (e) intracerebral pressure; therefore, the PLC may be connected to a subarachnoid pressure sensor 44; and (f) air or fluid flow through the control valves to establish the predetermined sequence, (8) a heating/cooling unit 80 connected to the reservoir 70 for controlling the temperature of the air or fluid in the reservoir; and (9) a ventilator 90 for incorporating pharmaceuticals for aerosolization in reservoirs R1, R2, and R3, connected via a positive-pressure air-delivery tube 91 to an endotracheal tube 92 defining an inspiratory/expiratory path to/from the patient 10; alternatively, the ventilator 90 may be connected via a positive-pressure air-delivery tube 91 to a T-piece 200 defining an inspiratory/expiratory path to/from the patient 10 connected to an endotracheal tube 92, and an expiratory-only path connected to a mucous trap 201 outputting via connector 92A containing a flow rate sensor 202 to a glottal valve 203 and voice box 204.

The set of pressure cuffs include: a forearm cuff 20 for each forearm region 11, a lower leg cuff 21 for each lower leg region 12, an upper arm cuff 22 for each upper arm region 13, a thigh cuff 23 for each thigh region 14, an abdominal cuff 24 for the abdomen region 15, and a thoracic cuff 25 for the thorax region 16. While this embodiment uses air to pressurize the cuffs, it is intended that any pressurizing medium, gas, gel, or liquid, may be used. Furthermore, while this embodiment includes a set of pressure cuffs interconnected by embedding each cuff in a fabric or mesh clothing unit or by connecting each cuff with a system of straps and stirrups, it is intended that the pressure cuffs may be independently attached to different body regions. Still furthermore, while this embodiment includes separate sets of supply lines 31A-35A and return lines 31B-35B, it is intended that a single set of supply/return lines that both carry the pressurizing medium to the set of pressure cuffs and return the pressurizing medium to the reservoir may be used. Moreover, while this embodiment uses a PLC including a software application to operate the control valves and to establish a pressuring-up sequence and to establish full cybernetic control of respiration, it is intended that any conventional controller mechanism can be used to achieve the desired sequence and control. Also, it is intended that other embodiments may be fabricated to accommodate different body shapes and sizes including, but not limited to, male or female, child or adult, and amputees or individuals missing one or more limbs.

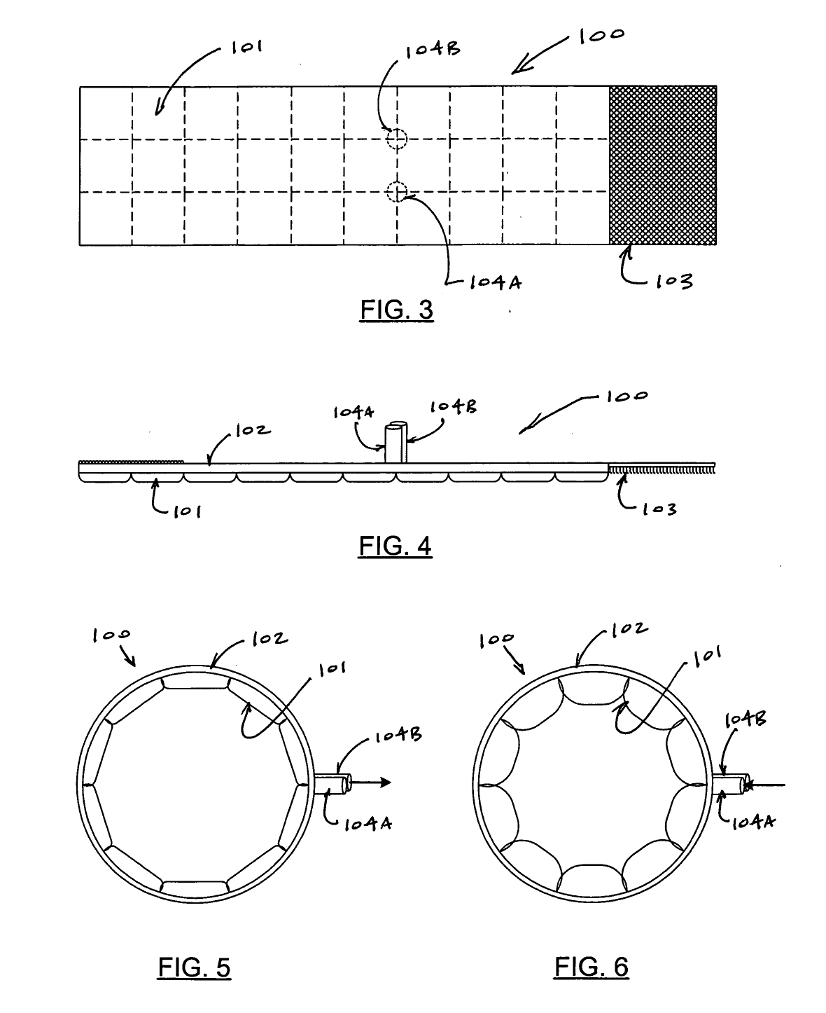

With respect to FIGS. 3-6, each pressure cuff 100 comprises a set of bladders 101 for receiving air to compress a body region, a band 102 for holding the bladders together, an attaching means 103 for attaching the band to a particular body region, and an input port 104A for receiving air to fill the set of bladders and an output port 104B for expelling air from the set of bladders back to the reservoir. While this embodiment uses a Velcro (or its equivalent) attaching means, it is intended that any conventional attaching means may be used including, but not limited to, buckles, laces, elastic bands, adhesives and buttons. Furthermore, while this embodiment includes a pressure cuff with each bladder in the set of bladders having its own input port and output port such that each bladder may be filled and expelled with a pressurizing medium independently, it is intended that there may also be only one input port and one output port for the entire set of bladders.

With respect to FIG. 2, the PLC 60 is connected to a series of measuring and sensor devices to collect patient response feedback, including: (1) a thermistor probe 41 and a patient-controlled thermostat 42 to facilitate temperature control of the patient; (2) transcutaneous oxygen sensors, carbon dioxide sensors, and arterial blood gas sensors and pH sensors 43 for facilitating control of respiratory rate, tidal volume, and inspired oxygen concentration; and (3) a subarachnoid pressure sensor 44 for facilitating control of intracerebral pressure.

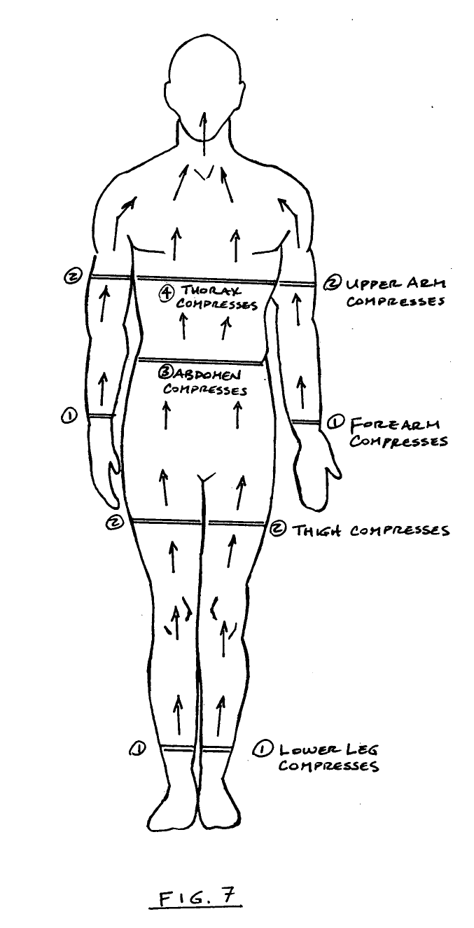

In operation, the external pressure garment of the present invention facilitates expiratory respiration in the patient and results in improved oxygen supply to the brain via blood flow from the body extremities of the patient (i.e., arms and legs) to the head. This is achieved by first strapping the pressure cuffs 20-25 onto the patient 10 at the locations 11-16 as depicted in FIG. 1. Once the pressure cuffs are secured, a PLC (e.g., a computer and software application) is used to establish a pressuring sequence for the cuffs. This sequence is illustrated in FIG.7. First, the forearm cuffs and lower leg cuffs are pressured up to compress the forearm and lower leg respectively. Second, the upper arm cuffs and thigh cuffs are pressured up to compress the upper arms and thighs respectively. Third, the abdominal cuff is pressured up to compress the abdomen. Finally, the thoracic cuff is pressured up to compress the thorax. This pressuring sequence serves to externally compress the lungs to assist the patient with expiratory functionality and improves the flow of oxygen-enriched blood to the brain.

The steps of pressurizing of the cuffs in accordance with the present invention are accomplished by an air pump 50 supplying air from a reservoir 70 to the pressure cuffs via a set of supply lines 3 1A-35A and a central processor 40 establishing each flowpath by means of a set of control valves as depicted in FIG. 2. For example, to pressure up the forearm cuff 20, the PLC 60 issues a command to the set of control valves 40 to open a flowpath from the reservoir 70 though the forearm supply line 30A. This enables the air pump 50 to inflate the bladder of the forearm cuff 20 and apply external pressure to the forearm of the patient. (See also FIG. 6). Of course, simultaneously, the lower leg cuffs are being pressured up. Once the pressuring sequence advances to the upper arm/thigh cuffs, to the abdominal cuff, and finally to the thoracic cuff, the PLC 60 issues a command to the set of control valves 40 to open a flowpath from the forearm return line 30B back to the reservoir 70. Air return is accelerated by the vacuum side of the air pump. This permits the bladder of the forearm cuff 20 to rapidly deflate and release the pressure from the forearm of the patient. (See also FIG. 5). This process of inflation and deflation is repeated for each pressure cuff along the pressuring sequence. As the air or fluid is circulated for pressurization purposes, it is heated or cooled by the heater/cooler 80 according to the patient's thermostat setting or according to the clinician's override. Thermistors for temperature monitoring and control may be inserted preferentially internally transesophageally or rectally, or, in an alternative embodiment, cutaneously. Clinicians may select hypothermic or hyperthermic treatment modalities.

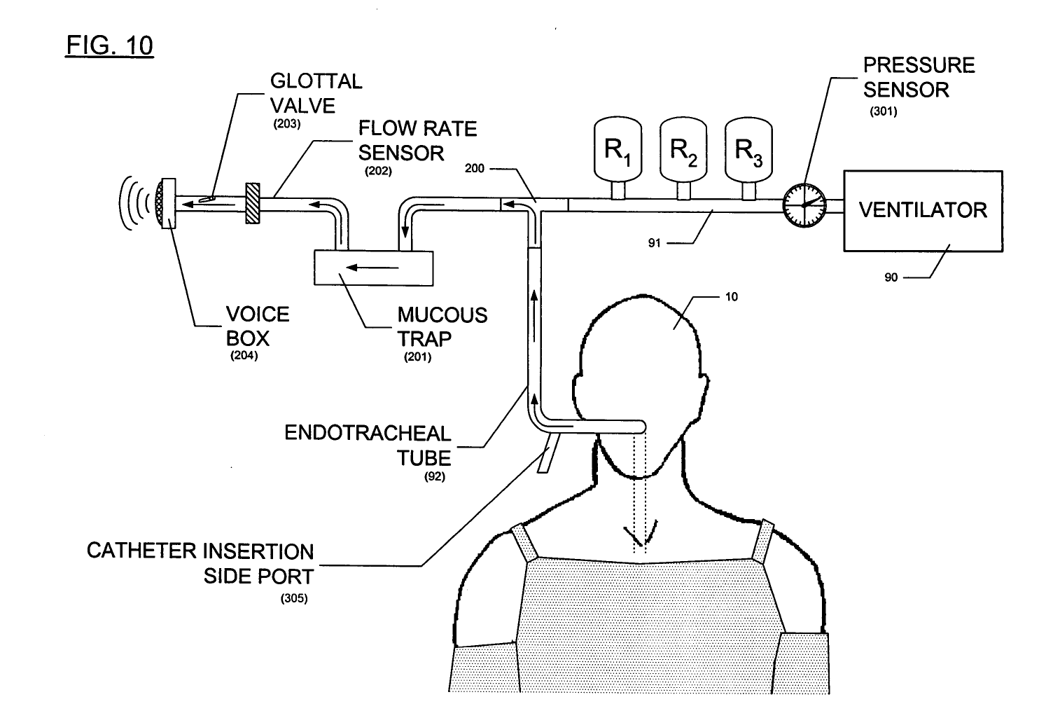

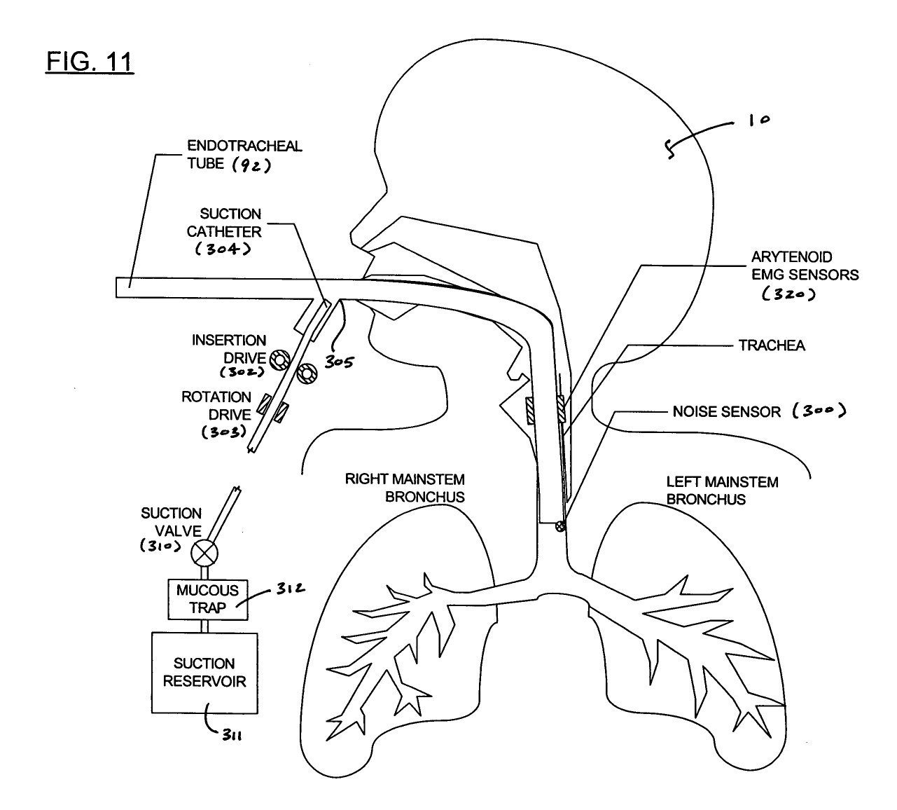

In a preferred embodiment of the present invention, the external pressure garment is used in combination with a positive pressure ventilator such that complete respiratory control—both expiratory and inspiratory functions—of the patient can be achieved. Paired in that fashion, as the air or fluid is being pumped into the cuffs of the external pressure garment, initiating the expiratory cycle, the glottal valve 203 opens to allow normal expiration (See FIG. 10). After the air or fluid has been pumped from each cuffof the external pressure garment, completing the expiratory cycle, the glottal valve 203 closes, and the PLC 60 triggers the ventilator 90 to provide positive pressure inspiration (See FIG. 8). To initiate startup, the PLC 60 accepts height, weight, and gender data and selects default tidal volume, respiratory rate, and inspired oxygen concentration, or else these settings may be entered manually. The PLC 60 monitors inspiratory pressure, transcutaneous or intraarterial oxygen, carbon dioxide, and pH data, and adjusts tidal volume, respiratory rate, and inspired oxygen concentration to meet clinician-selected parameters. The PLC 60 also monitors expiratory pressure and flow rate and adjusts cuff pressure. The PLC 60 also initiates therapeutic trials of aerosolized pharmaceuticals to lower expiratory and inspiratory pressure when indicated, trialing agents individually and in combination to achieve best effect.

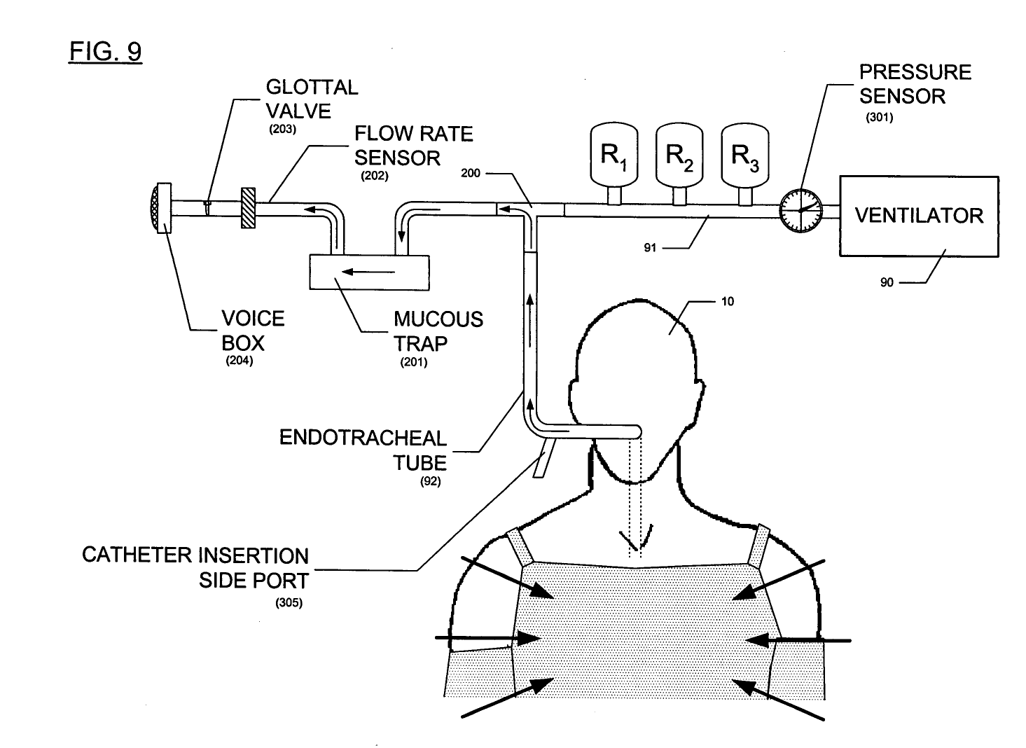

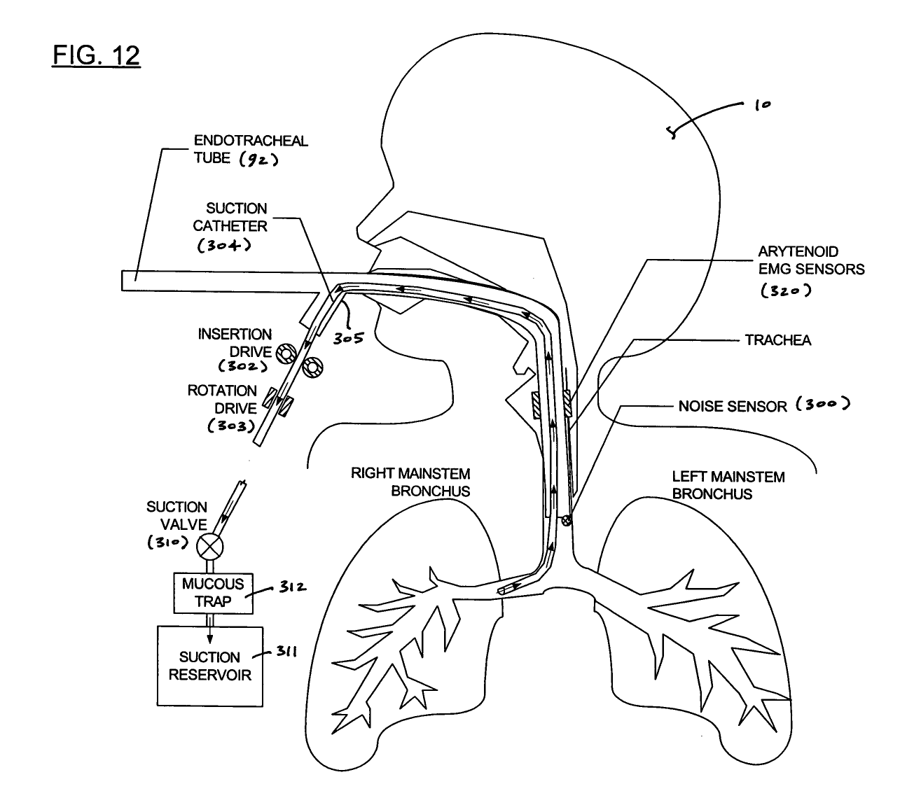

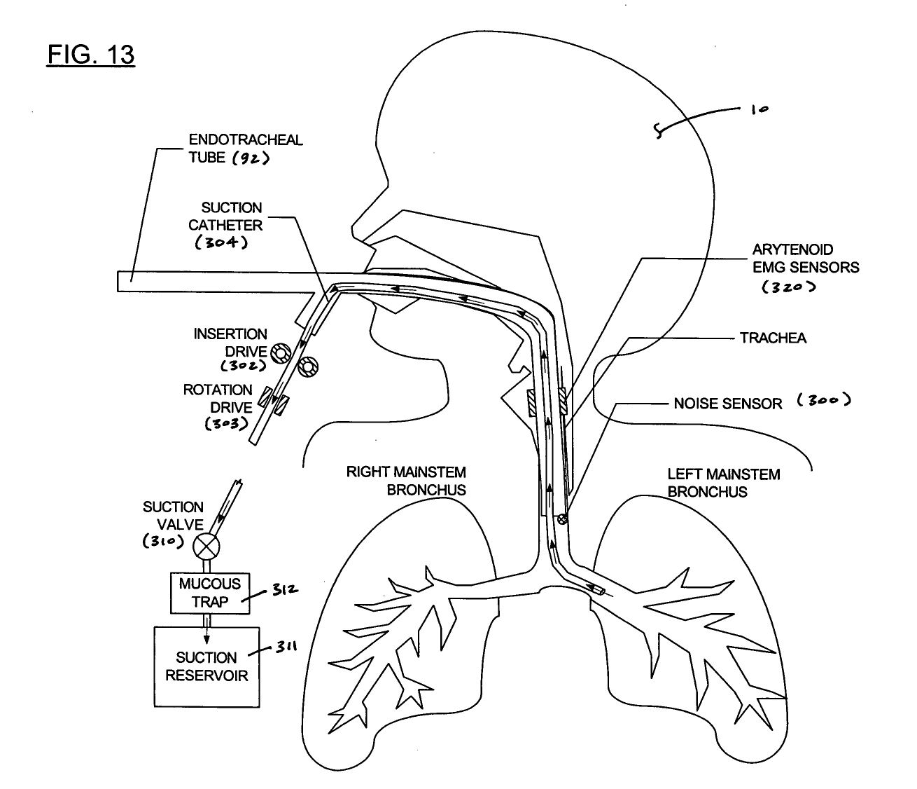

With respect to FIGS. 8-13, in another embodiment of the present invention, the PLC further monitors noise in the upper airway and initiates “cough” functionality and “suction” functionality when indicated. While a periodic cough/suction interval may be set by the clinician, ordinarily the noise sensor 300 (e.g., a microphone) detects increased upper airway noise, or else the pressure sensor 301 detects increased inspiratory pressure. In either case, at the beginning of the next expiratory cycle, the glottal valve 203 remains closed until expiratory pressure has increased to an appropriate level for effective coughing (as shown in FIG. 9). The glottal valve 203 is then opened, allowing the rapid egress of air effecting the cough (as shown in FIG. 10). Immediately after a cough, the PLC directs the insertion motor 302 to drive the soft curved suction catheter 304 positioned within the airtight side port 305 down into the right mainstem bronchus, whereupon suction valve 310 opens to vacuum as the insertion motor retracts the catheter (as shown in FIG. 12). The valve 310 is then closed, and the roll-control motor 303 rolls the catheter 304 approximately 180 degrees. The insertion motor 302 then inserts the catheter 304 into the left mainstem bronchus and the suction process is repeated (as shown in FIG. 13). After suction is completed, the roll-control motor 303 rolls the catheter 304 back into its resting position (as shown in FIG. 11). As the PLC monitors the above parameters, it provides continuous instantaneous and trend graphical displays of heart rate, EKG, respiratory rate, temperature, arterial pH or oxygen saturation, and parameters of the Henderson-Hassalbach equation. It will also display a short narrative interpretation of acid-base status including suggestions for differential diagnosis and treatment considerations. Because the PLC is continuously monitoring acid-base status, it will automatically provide respiratory compensation for metabolic acid-base disturbances. Moreover, because the PLC is continuously monitoring subarachnoid pressure, it will alter carbon dioxide status to accommodate lowering of elevated subarachnoid pressure. Finally, because the PLC 60 is continuously monitoring all of the above input parameters, and altering its outputs for each respiratory cycle, it will automatically produce cycle-to-cycle variability, a highly desired outcome, without the use of artificial variability protocols. Another embodiment of the PLC also accepts electroencephalography (“EEG”) input, allowing incorporation of brain activity analysis in setting its output levels.

In an alternative embodiment of the present invention, the glottis 203 may function to remain closed at the end of inspiration without expiration ensuing, and thus breath-holding is accomplished.

In yet another embodiment of the present invention, the thoracic pressure cuff 25 (FIG. 1) includes a set of integrated EKG leads for connection to a heart monitoring device. It is intended that the EKG leads may provide for any standard connection including, but not limited to, 3-lead and 12-lead outputs, and augmented output connectors for connection with multiple-lead EKG machines.

In a further embodiment of the present invention, the system not only outputs the EKG data to the clinician, but also uses the information for gating expiratory garment pressure so as to minimize interference to arterial flow.

In still another embodiment of the present invention, the thoracic pressure cuff 25 (FIG. 1) includes an integrated pair of anterior and posterior plates to facilitate defibrillation. The plates may be accessed by a standard manually operated defibrillator pad, an automatic external defibrillator device, or an integrated automatic external defibrillator device.

In a further embodiment of the present invention, the thoracic pressure cuff 25 (FIG. 1) includes a set of integrated piezo vibrators or other vibration devices for facilitating pulmonary toilet.

With respect to FIG. 11, in still a further embodiment of the present invention, the PLC is connected to (1) laryngeal electromyleogram (“EMG”) sensors 320 (i.e., speech recognition pick-ups) on the sides of the endotracheal tube 92, (2) lingual EMG sensors 321 on the ventral surface of the endotracheal tube 92, and (3) facial EMG sensors 322 all near the arytenoid cartilage of the larynx of the patient. The PLC collects and passes this EMG information to the glottal valve 203 and voice box 204 (FIG. 8). The glottal valve 203 and voice box 204 receives input from the laryngeal 320, lingual 321, and facial 322 EMG sensors to effect speech as described herein. In an alternative embodiment, the same EMG sensors 320, 321, and 322 may provide input for an electronic or electromechanical vocal apparatus to effect completely artificial speech.

In yet another embodiment of the present invention, the expiratory path distal to the voice box 204 leads to a carbon dioxide scrubber, filtration device, dialysis device, or other purification device for recycling of the gas or liquid previously delivered to the subject by the ventilator and subsequently expelled by the external pressure garment. After such processing, the processed gas or liquid is returned to the ventilator for reuse via a recycling path.

In another embodiment of the present invention, any standard cuff may be substituted with a transparent version to allow visual monitoring of the underneath body part. Alternatively, the pressure cuffs may have Velcro flaps that may be removed for nursing access to sites of interest.

In a further embodiment of the present invention, the thoracic cuff may be available in a variety of cup sizes to accommodate various breast shapes, so that a better fit may achieved to help minimize compressive pain.

In another embodiment of the present invention, the pulmocardiac assist system maybe wall-mounted (or otherwise-mounted) in any room both in patient-care settings and in residences. Alternatively, the pulmocardiac assist system may be mounted on various portable devices such as carts, self-powered carts, wheelchairs, automobiles, or beds.

In yet another embodiment of the present invention, the pulmocardiac assist system may be incorporated into an underwater diving pressure suit, a space suit, or a high-performance jet aircraft. In all of these embodiments, the system may cause respiration with either a gas or liquid; but for amelioration of the effects of acceleration, a liquid inspirate is preferred.

In still another embodiment of the present invention, the pulmocardiac assist system may be employed to achieve full-liquid breathing. Since the ventilator is capable of pumping both gases and liquids (such as fluorocarbons), full-liquid breathing may be facilitated. The heretofore exhausting work of expiring liquid from the lung is overcome by the external pressure garment. Therefore, the known benefits of liquid breathing—including, but not limited to, acceleration tolerance, pulmonary lavage, hyperoxygenation, and surfaction—are easy to tolerate for unlimited periods of time. This makes possible new applications for deep-sea diving depth tolerance, jet aircraft hyper-maneuverability, and spacecraft hyper-acceleration. The latter two applications are additionally facilitated by filling the cockpit, or an acceleration-reinforced sub-compartment of the cockpit, with the same fluid that is being pumped by the ventilator. The subject in these circumstances is immersed in the medium, and at the same time breathes it. The expiratory path is easily configured for both open-circuit and closed-circuit design, so that recirculation/recycling is possible whenever desired.

In an alternative embodiment of the present invention, the external pressure garment is employed for use by a vehicle operator or passenger (e.g., a race car driver, or a person traveling in a very high velocity vehicle) as a personal airbag system. This embodiment includes an interconnected body suit, headpiece, and neckpiece which include and employ pressure cuffs. The pressure cuffs provide complete coverage of the torso and limbs up to the wrists and ankles. The body portion would fasten to the cuffs which in-turn would attach to the outside of the wearer's helmet (of the type that completely covers the wearer's head and with only a visor) by way of the neck cuff that would have a custom-fit stiff inner ring that would protect the wearer's neck and trachea. The face shield of the helmet could be covered with pressure cuffs of a clear material (which could require the use of an air supply or even a positive pressure ventilator if needed) or could be left uncovered. The pressure cuffs are controlled by a modified logic controller. The logic controller includes inputs for receiving vehicular data critical to the safety of the operator/passenger wearing the personal airbag system. This data may include any conventional data calculated and managed by a vehicle such as speed/velocity rates and impact signals. This permits the logic controller to coordinate the reaction time and inflation rates the speed of the vehicle. The medium used to pressure the cuffs may include air, liquid or gas. The material from which the cuff is fabricated as well as the pressuring medium may have fire-retardant properties. Moreover, the personal airbag system could include an integrated pair of anterior and posterior plates to facilitate defibrillation by process of applying an electric shock to the heart of the subject.

While certain features and embodiments have been described in detail herein, it should be understood that the invention includes all of the modifications and enhancements within the scope and spirit of the following claims.

1. An apparatus for facilitating respiratory functions in a subject, comprising:
(a) a removable wrap adapted for encircling the torso portion of a body;
(b) a plurality of channels in the wrap for expanding and contracting the wrap in response to a stimulus;
(c) a drive system for stimulating the wrap to expand and contract the wrap for simulating respiration in the subject.

2. The apparatus of claim 1, wherein the wrap further includes a feedback system for monitoring the condition of the subject.

3. The apparatus of claim 2, wherein the feedback system comprises a set of sensors attached to the subject for monitoring cardiovascular vital signs.

4. The apparatus of claim 2, wherein the feedback system comprises a set of sensors attached to the subject for monitoring temperature.

5. The apparatus of claim 2, wherein the feedback system comprises a set of sensors attached to the subject for monitoring pH and blood gases.

6. The apparatus of claim 2, wherein the feedback system comprises a set of sensors attached to the subject for monitoring EEG and EKG and subarachnoid pressure.

7. The apparatus of claim 2, wherein the feedback system comprises a set of sensors and a set of probes attached to the subject for monitoring and optimizing ventilatory and heart rate parameters.

8. The apparatus of claim 1, wherein the wrap encircles the torso and extends from an upper terminus just below the armpits of the subject to a lower terminus beneath the diaphragm of the subject.

9. The apparatus of claim 1, wherein the wrap encircles the torso and extends from an upper terminus near the diaphragm of the subject to a lower terminus just above the groin of the subject.

10. The apparatus of claim 1, wherein the channels are a set of airtight chambers and wherein the drive system comprises a pump for pumping air into and out of the chambers.

11. The apparatus of claim 2, wherein the drive system is responsive to the feedback system to adjust the respiratory assist function dependent upon the monitored condition of the subject.

12. The apparatus of claim 1, wherein the wrap further comprises a garment, said garment comprising a plurality of pressure cuffs, said plurality of pressure cuffs comprising: (i) a set of pressure cuffs encircling the torso of the subject, and (ii) a set of pressure cuffs encircling the extremities of the subject.

13. The apparatus of claim 12, wherein the garment is transparent to allow visual access to selected body areas.

14. The apparatus of claim 12, wherein the pressure cuffs are elastic.

15. The apparatus of claim 12, wherein the plurality of pressure cuffs further include:
(a) forearm cuffs, extending approximately from wrist to elbow;
(b) leg cuffs, extending approximately from ankle to knee;
(c) arm cuffs, extending approximately from elbow to axilla;
(d) thigh cuffs, extending approximately from knee to groin;
(e) an abdominal cuff, extending approximately from pubis to subcostal margin; and
(f) a breast-conforming thoracic cuff, extending approximately from subcostal margin to infraclavicular area.

16. The apparatus of claim 12, wherein the garment is jointed to allow for full and ready access to the body at head, neck, hands, feet, elbows, knees, pelvic girdle, shoulder girdle, and thoraco-abdominal junction.

17. The apparatus of claim 12, wherein the garment is jointed and each cuff includes detachable cuff segments to allow full and ready access to any selected body part.

18. The apparatus of claim 12, wherein the thoracic cuff further includes embedded cutaneous electrical contacts for electrocardiography and defibrillation.

19. The apparatus of claim 12, wherein the thoracic cuff further includes embedded vibrating devices for pulmonary toilet.

20. The apparatus of claim 15, further including a securing system for preventing migration of the cuffs, said securing system comprising shoulder straps on the thoracic cuff, ankle stirrups on the lower leg cuffs, and straps connecting all cuffs to each other.

21. The apparatus of claim 12, wherein each pressure cuff further includes a set of expandable chambers for receiving a medium wherein the set of chambers expands to apply pressure to the body part enclosed by that cuff.

22. The apparatus of claim 21, wherein the medium is a gas.

23. The apparatus of claim 21, wherein the medium is an electroexpansile gel.

24. The apparatus of claim 21, wherein the medium is a liquid.

25. The apparatus of claim 21, wherein the medium is adapted to be temperature-conditioned.

26. The apparatus of claim 1, wherein the drive system is adapted for selectively applying pressure to different parts of the body of the subject for inducing the subject to breathe outward thereby expiring air from lungs of the subject.

27. The apparatus of claim 1, wherein the drive system is further adapted for promoting circulation of blood from the extremities of the subject to the head of the subject.

28. The apparatus of claim 1, wherein the drive system is further adapted to facilitate electro cardiographic gating for reducing impairment of arterial circulation by relieving increased preloading momentarily during a cardiac cycle.

29. The apparatus of claim 1, further including:
(a) a heated and cooled reservoir for storing a transportable medium at a chosen temperature to fill the channels in the wrap;
(b) a pump to supply the medium to the channels;
(c) a set of lines for transporting the medium from the reservoir to the channels; and
(d) a processing device for controlling the flow of the medium to each respective channel.

30. The apparatus of claim 29, further including a controller for accepting inputs from the various sensors for controlling the pump for managing the flow of the medium into and out of the channels.

31. The apparatus of claim 30, further including thermostatic controls for the heating and cooling of the medium.

32. The apparatus of claim 31, wherein the thermostatic controls include provision for deliberate hypothermia, hyperthermia, and appropriate responses to fever.

33. The apparatus of claim 30, further including a coordinated positive pressure inspiratory ventilator for allowing control of at least one simultaneously administered aerosolized pharmaceuticals for automatic control of airway resistance and automatic control of respiratory rate, tidal volume, and inspired oxygen concentration to achieve desired arterial pH and blood gas concentrations in the subject.

34. The apparatus of claim 33, further including an endotracheal tube for connecting the subject to an inspiratory path from the ventilator, said endotracheal tube having an airtight and autosealing side port for insertion of a suction catheter into the trachea of the subject.

35. The apparatus of claim 34, wherein the controller, in response to the received inputs, causes the suction catheter to insert into the trachea of the subject, suction the right mainstem bronchus of the subject, and withdraw from the subject.

36. The apparatus of claim 35, wherein the controller, in response to the received inputs, causes the suction catheter to rotate approximately 180 degrees, insert into the trachea of the subject, suction the left mainstem bronchus of the subject, and withdraw from the subject.

37. The apparatus of claim 34 wherein the endotracheal tube connects the subject to an expiratory path comprising an artificial glottis.

38. The apparatus of claim 37, wherein the controller, in response to the received inputs, causes the artificial glottis to: (i) close prior to expiration of the subject whereby pressure behind the artificial glottis increases to a predetermined level, and (ii) open once the predetermined pressure is reached thereby achieving a cough.

39. The apparatus of claim 37, wherein the controller, in response to received inputs, causes the artificial glottis to: (i) close at the end of inspiration and remain closed for a predetermined period of time wherein the subject may be transferred from the apparatus to another device for respiratory assistance.

40. The apparatus of claim 37, further comprising:
(a) a voice box connected to the expiratory path distal to the artificial glottis; and
(b) a set of EMG sensors attached to the endotracheal tube for receiving and sending arytenoid information to the artificial glottis and voice box whereby said voice box converts the arytenoid information into recognizable vocalization to facilitate speech while the subject is intubated.

41. The apparatus of claim 37, further comprising:
(a) an electronic or electromechanical voice synthesis device voice box connected to the expiratory path distal to the artificial glottis; and
(b) a set of EMG sensors attached to the endotracheal tube for receiving and sending arytenoid information to the artificial glottis and voice synthesis device whereby said voice synthesis device converts the arytenoid information into recognizable vocalization to facilitate speech while the subject is intubated.

42. The apparatus of claim 15, wherein the thoracic cuff includes a set of electrocardiogram leads.

43. The apparatus of claim 42, wherein the thoracic cuff further includes an integrated pair of anterior and posterior plates to facilitate defibrillation by process of applying a manually or automatically applied electronic shock to the heart of the subject.

44. The apparatus of claim 43, wherein the thoracic cuff still further includes a set of integrated vibration devices for facilitating pulmonary toilet in the subject.

45. The apparatus of claim 1, wherein said apparatus is adapted to facilitate portability of the subject.

46. The apparatus of claim 45, wherein said apparatus is adapted to fit into a fully-configured patient bed.

47. The apparatus of claim 45, wherein said apparatus is adapted to fit into a fully-configured manual and/or powered cart.

48. The apparatus of claim 45, wherein said apparatus is adapted to fit into a fully-configured automobile.

49. The apparatus of claim 45, wherein said apparatus is adapted to fit into a wall mounting.

50. The apparatus of claim 1, further comprising:
(a) a ventilator for supplying breathable air to the subject to facilitate inspiration;
(b) a purification device for processing expired air from the subject to remove carbon dioxide and other expired waste to produce a cleansed air;
(c) an inspiratory path from the ventilator to the subject to deliver the breathable air;
(d) an expiratory path from the subject to the purification device to deliver the expired air; and
(e) a recycle path from the purification device to the ventilator to deliver the cleansed air.

51. The apparatus of claim 1, further comprising:
(a) a ventilator for supplying breathable liquid to the subject to facilitate inspiration;
(b) a purification device for processing expired liquid from the subject to remove carbon dioxide and other expired waste to produce a cleansed liquid;
(c) an inspiratory path from the ventilator to the subject to deliver the breathable liquid;
(d) an expiratory path from the subject to the purification device to deliver the expired liquid; and
(e) a recycle path from the purification device to the ventilator to deliver the cleansed liquid.

52. The apparatus of claim 50, wherein said apparatus being arranged for use as a diving and/or pressure suit in an underwater environment.

53. The apparatus of claim 50, wherein said apparatus being arranged for use as a G-suit in an aerial and/or outer space environment.

54. The apparatus of claim 51, wherein said apparatus being arranged for use as a diving and/or pressure suit in an underwater environment, said apparatus for enclosing the subject within a reservoir of similar fluid as the breathable liquid.

55. The apparatus of claim 51, wherein said apparatus being arranged for use as a G-suit in an aerial and/or outer space environment, said apparatus for enclosing the subject within a reservoir of similar fluid as the breathable liquid.

56. The apparatus of claim 1, wherein said apparatus being arranged for use as a G-suit in a cockpit of an aerial vehicle.

57. The apparatus of claim 33, wherein said apparatus is adapted to facilitate portability of the subject.

58. The apparatus of claim 33, wherein said apparatus being arranged for use as a G-suit in a cockpit of an aerial vehicle.

59. An apparatus for facilitating respiratory and circulatory functions in a patient, comprising:
(a) a garment of pressure cuffs to be adorned by the patient, said garment comprising: (i) a breast-conforming thoracic cuff extending approximately from subcostal margin to infraclavicular area, (ii) an abdominal cuff extending approximately from pubis to subcostal margin, (iii) a set of forearm cuffs, each forearm cuff extending approximately from wrist to elbow, (iv) a set of lower leg cuffs, each lower leg cuff extending approximately from ankle to knee, (v) a set of upper arm cuffs, each upper arm cuff extending approximately from elbow to axilla, and (vi) a set of thigh cuffs, each thigh cuff extending approximately from knee to groin; each of said cuffs comprising a set of bladders for receiving an expanding medium;
(b) a feedback system comprising a set of sensors attached to the patient for monitoring patient-related variables;
(c) a drive system comprising a pump, a reservoir for holding the expanding medium, and a set of flow lines for connecting reservoir to each of the pressure cuffs via the pump and a set of control valves, said drive system for delivering the expanding medium to the pressure cuffs in accordance to a pressuring sequence; and
(d) a controller system comprising a PLC, said controller system connected to the feedback system and the drive system, said controller system for establishing the pressuring sequence, said sequence determined by analysis of the patient-related variables monitored by the feedback system.

60. The apparatus of claim 59, wherein the thoracic cuff further includes an integrated pair of anterior and posterior plates to facilitate defibrillation by process of applying an electronic shock to the heart of the patient

61. The apparatus of claim 59, wherein the patient-related variables to be monitored by the feedback system are selected from the group consisting of: cardiovascular vital signs, temperature of the patient, pH and blood gas levels, EEG, EKG, and subarachnoid pressure, ventilatory parameters, arytenoid EMG information from the larynx of the patient, and pulmonary noise.

62. The apparatus of claim 61, wherein the thoracic cuff further includes an integrated pair of anterior and posterior plates to facilitate defibrillation by process of applying an electronic shock to the heart of the patient.

63. The apparatus of claim 59, further comprising a heating and cooling device connected to the reservoir of expanding medium, said heating and cooling device for managing the temperature of the expanding medium pursuant to directions from the controller system.

64. The apparatus of claim 63, wherein the thoracic cuff further includes an integrated pair of anterior and posterior plates to facilitate defibrillation by process of applying an electronic shock to the heart of the patient.

65. The apparatus of claim 59, further comprising:
(a) a ventilator for providing inspiratory functions to the patient; and
(b) an endotracheal tube for connecting the patient to ventilator, said endotracheal tube comprising a port for receiving a suction catheter and a T-piece for defining an inspiratory-only path from the ventilator to the patient and an expiratory-only path from the patient to an outlet.

66. The apparatus of claim 65, wherein the thoracic cuff further includes an integrated pair of anterior and posterior plates to facilitate defibrillation by process of applying an electronic shock to the heart of the patient.

67. The apparatus of claim 65, wherein the ventilator is a coordinated positive pressure inspiratory ventilator for allowing control of aerosolized pharmaceuticals for control of airway resistance and automatic control of respiratory rate, tidal volume, and inspired oxygen concentration to achieve desired arterial pH and blood gas concentrations in the patient.

68. The apparatus of claim 65, wherein the controller system, in response to the patient-related variables monitored by the feedback system, causes the suction catheter to insert into the port of the endotracheal tube to facilitate pulmonary suctioning of the patient.

69. The apparatus of claim 65 wherein the outlet of the expiratory-only path comprises a glottal valve.

70. The apparatus of claim 69, wherein the controller system, in response to the patient-related variables monitored by the feedback system, causes the glottal valve to: (i) close prior to expiration of the patient whereby pressure behind the glottal valve increases to a predetermined level, and (ii) open once the predetermined pressure level is reached thereby achieving a cough.

71. The apparatus of claim 59, wherein the control system, in response to the patient-related variables monitored by the feedback system, causes arytenoid information to be sent to the glottal valve to facilitate speech while the patient is intubated.

72. The apparatus of claim 59, wherein the pressuring sequence comprises the following order: (i) first, pressurization of the set of forearm cuffs and the set of lower leg cuffs, (ii) second, pressurization of the set of upper arm cuffs and the set of thigh cuffs, (iii) pressurization of the abdominal cuff, and (iv) pressurization of the breast-conforming cuff.

73. Personal airbag apparatus for use by a person in a vehicle wearing a helmet, comprising:
(a) a body suit comprising a set of pressure cuffs, said set of pressure cuffs including: (i) a pressure cuff for encircling the torso, and (ii) pressure cuffs for encircling the extremities of the person up to the wrists and ankles;
(b) a headpiece comprising a set of pressure cuffs, said headpiece formed to fit over the helmet of the person;
(c) a neckpiece comprising a set of pressure cuffs, said neckpiece formed to encircle the neck of the person, said neckpiece including a custom-fit stiff inner ring to protect the trachea and neck of the person, said neckpiece for connecting the body suit and headpiece; and
(d) a logic controller for receiving input data from the vehicle and stimulating the pressure cuffs of the body suit, headpiece, and neckpiece to expand and contract according to the data.

74. The personal airbag apparatus of claim 73, wherein the set of pressure cuffs of the headpiece are fabricated from a transparent material such as not to impede vision of the person.

75. The apparatus of claim 73, wherein the input data received by the logic controller includes vehicular speed information for coordinating reaction time and inflation rates with the speed of the vehicle.

76. The apparatus of claim 73, further comprising a pressurizing medium to facilitate contraction of the pressure cuffs, said pressuring medium having fire retardant properties.

77. The apparatus of claim 73, wherein the set of pressure cuffs of the body suit, the headpiece, and the neckpiece have fire retardant properties.
